(19) United States
(12) Patent Application Publication
(10) Pub. No.: US 2009/0018325 A1
(43) Pub. Date: Jan. 15, 2009

(54) PROCESS FOR PREPARING L-NUCLEIC ACID DERIVATIVES AND INTERMEDIATES THEREOF

(76) Inventors: Jacques Cercus, Rixheim (FR); Michael Foulkes, Riehen (CH); Thomas Heinz, Breitenbach (CH); Daniel Niederer, Oberwil (CH); Beat Schmitz, Allschwil (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/281,630

(22) PCT Filed: Mar. 15, 2007
(86) PCT No.: PCT/EP2007/052464

§ 371 Date: Sep. 4, 2008

Related U.S. Application Data
(60) Provisional application No. 60/782,604, filed on Mar. 15, 2006.

(51) Int. Cl. C07H 19/00

(52) U.S. Cl. 536/28.54

(57) ABSTRACT

A novel method has been found to produce 2,2′-anhydro-1-(β-L-arabinofuranosyl)thymine as a novel useful intermediate compound. A novel method has been further found to produce thymidine from 2,2′-anhydro-1-(β-L-arabinofuranosyl)thymine. According to these methods, synthesis of various L-nucleic acid derivatives, synthesis of which has been difficult till now, is possible.

### FIELD OF THE INVENTION

The invention relates to an improved process for the synthesis of L-nucleic acid derivatives useful as a medicine, as well as to synthesis of intermediates therefor.

### BACKGROUND

Recently, L-nucleic acid derivatives have been sought for their desirable effects as medicines. However, L-nucleic acid derivatives are unnatural products and raw materials to produce the same do not substantially occur in nature. L-arabinose has generally been used as a raw material in synthesis of L-nucleic acid derivative. Various processes starting with L-arabinose have proven to be long and complex steps to conduct industrially under a safe and cost efficient basis (see, for example, Nucleosides & Nucleotides, 18(2), 187-195 (1999); Nucleosides & Nucleotides, 18(11), 2356 (1999)).

Thymidine derivatives have been developed through use D-nucleic acid intermediates such as 2,2′-anhydro-1-(β-D-arabinofuranosyl) (JP-A-6-92988; JP-A-2-59598, J. Org. Chem., 60(10), 3097 (1995)). L-nucleic acid intermediates have also been used such as in EP1348712, U.S. Pat. No. 4,914,233 and WO03/087118.

Yet these processes do not meet the most cost efficient and straightforward level of industrial applicability.

Mitsui Chemicals Inc., reported methods for preparing 2,2′-anhydro-1-β-L-arabinosfuranosyl)thymine and 2,2′-anhydro-5,6-dihydrocyclouridine, which are useful as intermediates in the synthesis of L-nucleic acids (PCT Publication No. WO 02/044194; EP 1348712 A1). The 7-step Mitsui process includes:

(a) reacting L-arabinose with cyanamide to provide L-arabinoaminooxazoline (1)

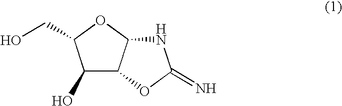

(b) reacting L-arabinoaminooxazoline (1) with an acrylic acid derivative (2)

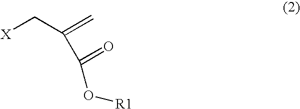

(wherein R1 is a lower alkyl group, and X is bromine, mesylate or acetate derivative, chlorine, a p-toluenesulfonyloxy group or a methanesulfonyloxy group) to synthesize a L-arabinoaminooxazoline derivative (3)

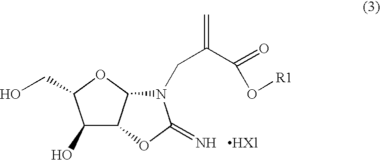

(wherein X and R1 have the same definitions as given above),
(c) reacting a base with the L-arabinoaminooxazoline derivative (3) to synthesize an L-2,2′-anhydronucleic acid derivative (4)

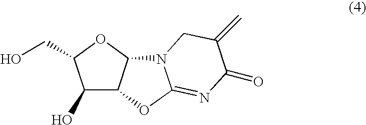

(d) isomerizing the L-2,2′-anhydronucleic acid derivative (4) to synthesize 2,2′-anhydro-1-β-L-arabinofuranosyl)thymine (5)

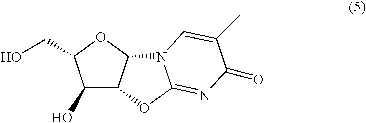

(e) subjecting the 2,2′-anhydro-1-(β-L-arabinofuranosyl)thymine (5) either to halogenation and subsequent protection, or to protection and subsequent halogenation, or to simultaneous halogenation and protection, to form (6)

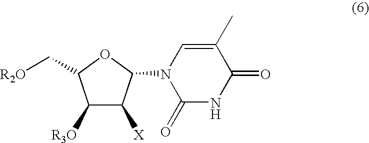

(wherein R2 and R3 are each independently a protecting group for hydroxyl group and X is a halogen),
(f) dehalogention of (6) to (7) and

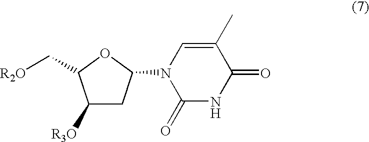

(g) deprotection of compound (7) to synthesize a β-L-thymidine (8)

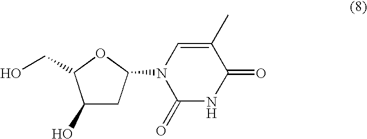

### SUMMARY OF THE INVENTION

As it is desirable to have a process that is more easily adapted to large scale production a novel efficient process for preparing β-L-thymidine (8) on large scale was developed and is disclosed herein.

Surprisingly, the present invention improves upon previous methods to produce L-2,2′-anhydronucleic acid derivatives. In one aspect, the cyclization and isomerization conditions to produce 2,2′-anhydro-1-β-L-arabinofuranosyl)thymine (5) were improved. As a consequence, isolation by crystallization is possible instead of by the prior art of purification by column chromatography which is not suitable for large scale production. Compound (6), which is thermally unstable and potentially mutagenic is not isolated in solid form but is handled as a solution in ethylacetate. The ethylacetate solution of (6) can be directly used in the following hydrogenation step to form (7).

In another aspect, previous cyclization and isomerization conditions included addition of the cyclization solution, neutralized with acetic acid, to a suspension of palladium alumina in water at 80° C. in a hydrogen atmosphere. Experiments reveal that the reaction is extremely fast and that a major by-product is formed in increasing amounts with time. This by-product (formula A) originates from the hydrolysis of the product. The present invention significantly reduces the amount of by-products produced, increases the suitability for scale up and reduces the cost by controlling various parameters including the pH of the starting solution, lowering the temperature and significantly shortening the time required for mixing during the working temperature.

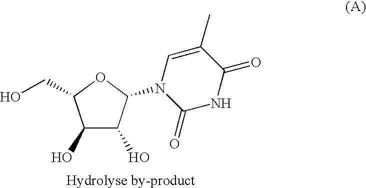

By reducing the working temperature another by-product previously neglected due to its apparent low amount was identified by LC-MS to be the product +2H, formula (B) below, as a diastereomeric mixture.

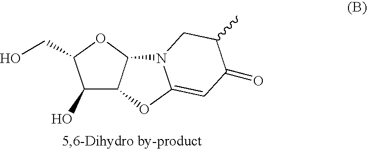

The structure of B was confirmed by synthesis. This by-product does not increase with the “hydrogenation” time and the formation can be explained by the hydrogenation of the exo-double bond in the starting material. The UV absorption of this by-product is five times weaker than that of the saturated product.

Isomerization works under hydrogen at any temperature; lower temperature decrease the hydrolysis and increase the amount of 5,6-dihydro by-product. The ratio of isomerization/hydrogenation is 80/20 at room temperature and approximately 95/5 at 65-80° C. At 65° C., an addition time of 1 hour and a stirring time of less than 1 hour is required to control hydrolysis to a level of less than 1%.

Various isomerization conditions were tested to reduce the competing hydrogenation and include;

Method 1) The catalyst suspension is activated in a hydrogen atmosphere. The hydrogen flow is maintained and the cyclization solution is added.
Method 2) The catalyst suspension is activated in a hydrogen atmosphere. The solution of the starting material 5 is added in an atmosphere containing a given amount of free H₂.
Method 3) The catalyst suspension is activated in a hydrogen atmosphere, then the reactor is purged with nitrogen to remove all free hydrogen. The cyclization solution is added under nitrogen.

In method 1, the catalyst (10% w/w) is suspended in water in a hydrogen flow for 15 min at room temperature. Then, the mixture is heated to the working temperature and the cyclization solution is added over 45-60 minutes at a constant temperature and under a slow hydrogen flow.

TABLE 1
Results (catalyst: Pd 5% on alumina)

| | % remaining starting | |
|---|---|---|
| | material | % dihydro by-product at |
| Temperature | 5 min after addition | reaction end (HPLC) |
| 25° C. | 20% (0% 60 min later) | 18% |
| 45° C. | 5% (0% 30 min later) | 9% |
| 55° C. | 0% | 6.5% |
| 65° C. | 0% | 4% |
| 75° C. | 0% | 2.7% |

A temperature greater than 60° C. is needed to minimize the amount of dihydro by-product formed. At this temperature the reaction is spontaneous and only requires stirring for a few additional minutes to complete the reaction. However, a temperature greater than 65° C. is not preferred as at higher temperatures (65 to 75° C.) some hydrolysis occurs. The main objective at 65° C. is to avoid hydrolysis and to maintain the reaction temperature during the addition. The addition time of the solution should be longer than 30 minutes to maintain the temperature during the addition of the cold solution. Other experiments at IT 65-75° C. show a low reproducibility concerning the dihydro by-product in which the amount varies between 4 and 10%. Parameters such as stirring speed and the amount of free/absorbed hydrogen can also play a role. Other catalysts: Pd on carbon, on BaSO₄, Pd(OH)₂, Rh on alumina have been tested but performed worse than Pd on alumina.

In method 3, the catalyst (10-30% w/w) is suspended in water under a hydrogen flow for 15 minutes at room temperature. Then the mixture is heated to the working temperature under hydrogen. The hydrogen flow is replaced by a nitrogen flow for 15 minutes and the cyclization solution is added over 45-60 minutes at a constant temperature and under a slow nitrogen flow.

TABLE 2
Results (catalyst: Pd 5% on alumina)

| | % remaining starting | |
|---|---|---|
| Temperature/amount | material | % dihydro by-product at |
| catalyst | 5 min after addition | reaction end (HPLC) |
| 70° C./10% | 24% | 3.7% |
| 70° C./20% | 5% | 3.2% |
| 70° C./25% | 0% | 2.9% |
| 55° C./25% | 0% | 3.0% |
| 45° C./25% | 0% | 2.6% |
| 35° C./25% | 31% (4% 40 min later) | 3.8% |

This isomerization works well in a nitrogen atmosphere but, as expected, a higher amount of catalyst is needed. At 70° C., with 10% catalyst, the conversion is only 76% and then, hydrogen has to be introduced to complete the reaction. The dihydro-by-product is still present, but in a rather lower and more reproducible amount of ˜3%. The results in the table have been obtained with a Pd/alumina catalyst

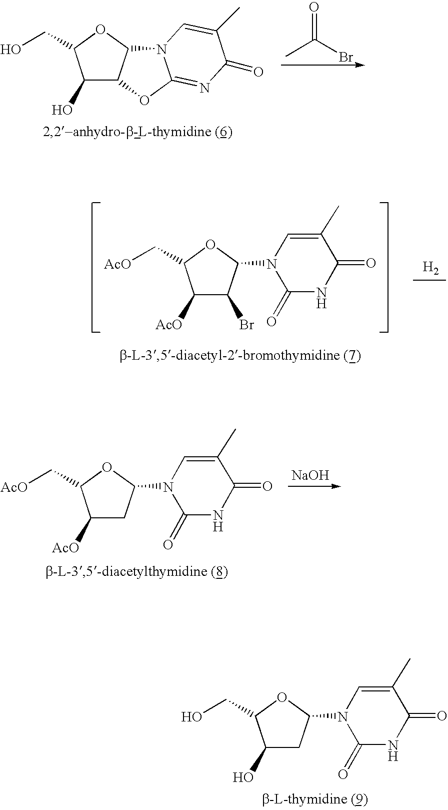

Surprisingly, the present invention improves upon previous methods to produce L-2,2′-anhydronucleic acid derivatives. Specifically, previous bromination and hydrogenation conditions included several solvent exchanges from ethyl acetate/DMF (bromination) to methanol (hydrogenation) and isopropyl alcohol (crystallization). DMF, which inhibits crystallization of (β-L-3′,5′-diacetyl-2′-bromothymidine), has to be removed by distillation or extraction to achieve acceptable yields of crystalline (β-L-3′,5′-diacetyl-2′-bromothymidine). DMF removal is difficult to realize on large scale because (β-L-3′,5′-diacetyl-2′-bromothymidine) it is not stable enough under the conditions to distill off DMF. It was surprisingly found that bromination and hydrogenation can both be achieved in ethyl acetate alone, avoiding change of solvents and isolation of the potentially mutagenic (β-L-3′,5′-diacetyl-2′-bromothymidine) in crystalline form.

For the success of the hydrogenation in ethyl acetate as solvent the presence of sodium acetate dissolved in water is essential. In dry ethyl acetate and in the presence of solid sodium acetate or other bases “by product” C formation is observed. FORMULA of by-product:

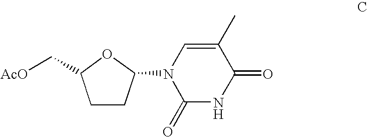

TABLE
Results of different hydrogenation experiments in ethyl acetate
Hydrogenation

| | | | | | | By- | |
|---|---|---|---|---|---|---|---|
| | | | Time | | Product | product C | |
| Katalyst | Base | Equiv. | 25° C. | | (HPLC) | (HPLC) | |
| Pd/Alox | Triethyl- | 1.0 | 14 | h | 88.7 | 8.6 | |
| 5% | amine | | | | | | |
| 15837/92 | | | | | | | |
| Pd/Alox | none | — | 21 | | trace | — | 94.4% |
| 5% | | | | | | | starting |
| 15334/14 | | | | | | | material |
| Pd/Alox | NaOAc × | 1 | 18 | | 82.1 | 9.5 | |
| 5% | 3 | | | | | | |
| 15334/50 | H₂O | | | | | | |
| | (solid) | | | | | | |
| Pd/Alox | 4% | 1 | 6 | | 95.4 | 1.3 | |
| 5% | NaOAc | | | | | | |
| 15349/16 | solution | | | | | | |
| Pd/Alox | 10% | 1 | | | | | |
| 5% | NaOAc | | | | | | |
| | solution | | | | | | |

### EXAMPLES

The present invention is described in more detail below by way of Examples. However, the present invention is in no way restricted thereto.

### Example 1

### Production of 2-amino-β-L-arabinofurano[1′,2′:4,5]oxazoline (2)

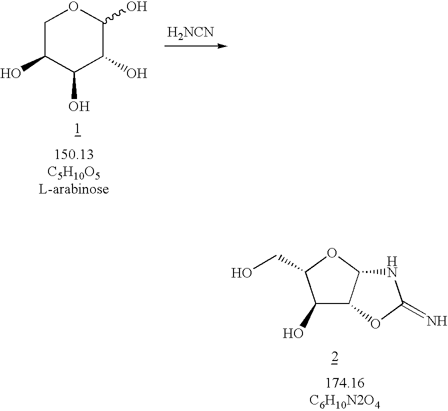

L-Arabinose (9 kg) is suspended in DMF (42.15 L) under stirring at room temperature and 50% cyanamide in water (6.25 kg) is added in 1 kg portions. During the addition an exotherm is observed and the temperature increases to 30° C. The suspension is warmed to 50 deg C. and is heated for 1 h. A solution of potassium carbonate, 28% in water (370.2 g) is added and the temperature increased to 60 deg C. for 8 h. During this time the mixture changes to a turbid beige solution and then crystallizes. After 8 h the reaction is cooled to 20 deg C. over 1 h and is kept at 20° C. for 10 h. Acetic Acid and ethyl acetate are added to the mixture drop wise over 45 minutes. The suspension is then cooled further to 0 deg C. and the product is isolated by filtration. The product 2 is washed with ethanol and dried in a vacuum oven at 45° C.

### Example 2

### Synthesis of ethyl 2-(chloromethyl)acrylate

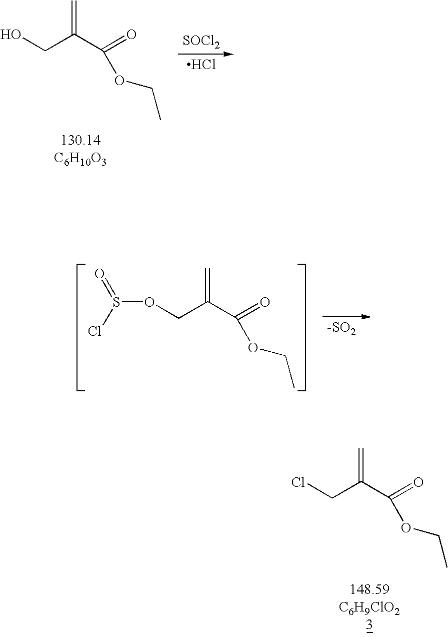

To ethyl (hydroxymethyl)acrylate (30.73 mol) under an inert atmosphere of nitrogen at 10° C. is added thionyl chloride (35.34 mol) drop wise keeping the internal temperature between 8-10° C. Upon completion of the addition the mixture is allowed to stir for an additional 15 minutes and then is slowly heated to 75° C. over 1 h. The mixture is kept at 75° C. for an additional 2 h and then heptane is added drop wise. The heptane is then distilled off in two portions removing the excess thionyl chloride. The crude chloride 3 is used directly in the next step.

### Example 3

### N-Alkylation of L-arabinoaminooxazoline to produce (3)

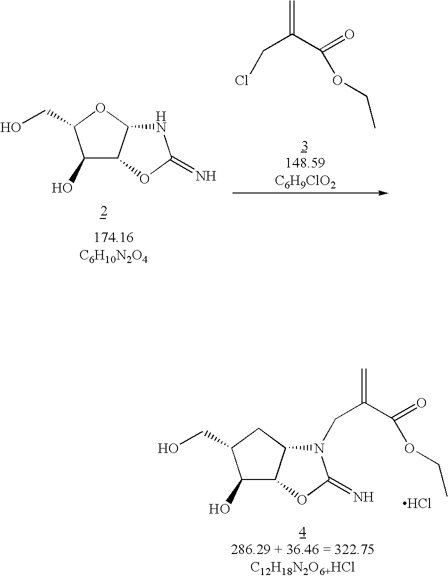

The crude chloride (3) from the previous reaction is dissolved in dimethylacetamide at 25° C. Compound 2 is added in portions and the resulting mixture is allowed to stir at room temperature for 4 h. Toluene is added drop wise over 10 minutes and the product slowly crystallizes. The mixture is stirred for 75 minutes at room temperature and an additional toluene is added and the mixture is allowed to stir overnight. The crystallized product is filtered and washed with Toluene/Ethanol 1:1. The product is dried in a vacuum oven at 45° C. overnight to afford compound 4 in 52.6% yield.

### Example 4

Cylclization of L-arabinoaminooxazoline (4) to produce an L-2-2′-anhydronucleic acid derivative 5 and isomerization of L-2-2′-anhydronucleic acid derivative to produce 2,2′-anhydro-1-(β-L-arabinofuranosyl)thymine (6)

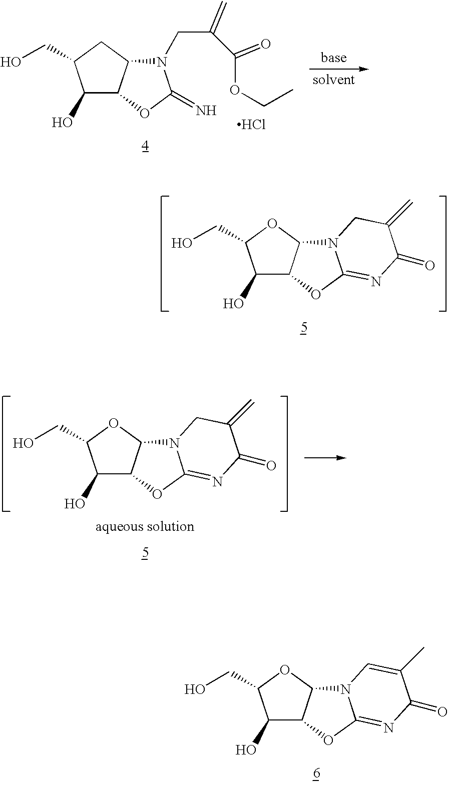

A solution of 4 and p-methoxyphenol in water is cooled to 8-10° C. in an ice bath. Potassium carbonate is added over one hour with stirring and the solution is cooled to 0-2 deg C. The resulting solution is allowed to stir for at least 4 hours. A 2 molar HCl solution is added drop wise keeping the temperature between 0 and 4° C. The solution is degassed with strong gas development and the pH of the resulting solution is approximately 6. The reaction mixture is stirred over night to afford an aqueous solution of 5.

In a separate vessel Pd on aluminum oxide (5%) is suspended in water under a nitrogen atmosphere. The vessel is purged with hydrogen for 10 minutes. Under the hydrogen atmosphere the mixture is heated to 60-65° C. over approximately 1 hour. The hydrogen flow is then stopped and the mixture is purged with nitrogen. To this suspension is added the aqueous solution of 5. keeping the temperature above 60° C. The reaction mixture is purged for another 10 minutes with hydrogen followed by an additional 2 minute purge with nitrogen. An additional purge cycle with nitrogen followed by hydrogen was performed. The batch was cooled to RT and purged again with nitrogen and filtered. The pH of the solution was adjusted with 2 molar aqueous HCl to approximately 6.5. The solvent was removed in vacuo to afford a slurry. Ethanol is added and the salts are filtered off. The filtrate was concentrated in vacuo, cooled to 0° C., and filtered to afford after drying white crystals of 6 in 74.3% yield.

### Example 5

### Synthesis of β-L-thymidine (9)

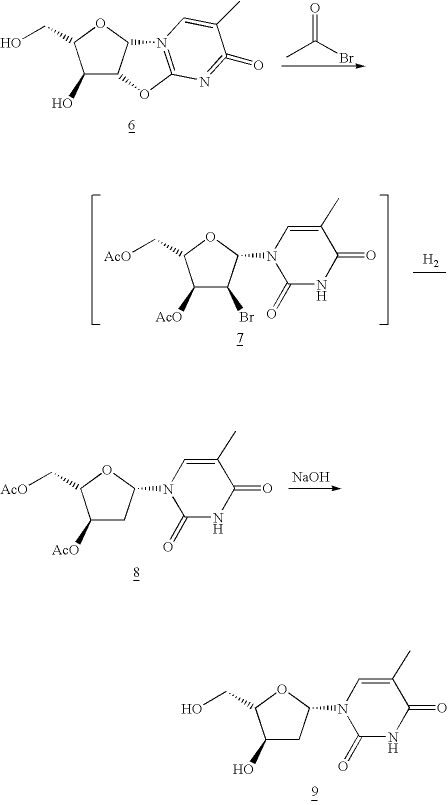

30.3 g of 2,2′-anhydro-1-(β-L-arabonfuranosyl thymine) derivative 6 is suspended at 25° C. in 150 ml ethyl acetate with 20.3 g dimethyl formamide (277 mmol). 34.1 g acetyl bromide (277 mmol) is added at 60° C. within 30 minutes. Stirring at 60° C. is continued for an additional 30 minutes. The mixture is then cooled to 25° C. IT and treated with aqueous potassium bicarbonate 25% until gas evolution is no longer observed (ca. 15 min). The phases are separated and the organic phase is washed with 20 ml aqueous sodium chloride solution (20%).

To the organic phase (containing β-L-3′,5′-diacetyl-2′-bromothymidine 7), a suspension of 5 g palladium/alox 5%, 10.33 g sodium acetate in 248 ml water is added and the resulting solution is hydrogenated at 25° C. for ca. 3 hours. The catalyst is filtered off and the aqueous phase is separated and extracted twice with 50 ml water. The combined water phases are extracted twice with 100 ml ethyl acetate. The combined organic phases are evaporated at 60° C. in vacuum. The oily residue obtained is dissolved at 70° C. in 230 ml of isopropyl alcohol. The resulting solution is seeded at 50° C. and stirred for ca. 1 hour. The suspension is cooled to −5° C. and stirred for two hours. After filtration and washing with cold isopropyl alcohol the product is dried at 60° C. overnight.

24.5 g β-L-3′,5′ diacetylthymidine 8 (75 mmol) and 1 g sodium hydroxide 30% (7.5 mmol) are heated for ca. 48 h in 90 ml ethanol at reflux. Then 0.53 g acetic acid (8.8 mmol) is added and the temperature is maintained at 76° C. for 30 minutes. The mixture is cooled to −5° C. The crude product 9 formed is filtered off, washed and dried at 60° C. overnight.

8.16 g β-L-thymidine crude (9) is dissolved in 101.2 g ethanol/water 93:7 (G/G) at reflux (78° C.). The solution is cooled to ca. 40° C. and a portion of solvent (approximately 68.5 g) is removed by distillation under vacuum. The suspension formed is cooled to 7° C. and stirred for one hour. The pure product is isolated by filtration, washed and dried at 60° C. in vacuo overnight.

We claim:

1. A process for producing L-thymidine comprising:
(a) a step of reacting L-arabinoaminooxazoline represented by the following formula (1) with an acrylic acid derivative represented by the following formula (2) (wherein R1 is a lower alkyl group, and X is chlorine, a p-toluenesulfonyloxy group or a methanesulfonyloxy group) to synthesize a L-arabinoaminooxazoline derivative represented by the following formula (3) wherein X and R1 have the same definitions as given above,
(b) a step of reacting a base with the L-arabinoaminooxazoline derivative represented by the formula (3) to synthesize a L-2,2′-anhydronucleic acid derivative represented by the following formula (4)
(c) a step of isomerizing the L-2,2′-anhydronucleic acid derivative represented by the formula (4) to synthesize 2,2anhydro-1-(β-L-arabinofuranosyl)thymine represented by the following formula (5)
(d) a step of subjecting the 2,2′-anhydro-1-(β-L-arabinofuranosyl)thymine represented by the formula (5) to halogenation and subsequent protection, or protection and subsequent halogenation, or protection and simultaneous halogenation to synthesize a 2′ position-halogenated L-thymidine derivative represented by the following formula (6) in solution,
wherein R2 and R3 are each independently a protecting group for hydroxyl group, with the proviso that said formula (6) compound is not isolated from said solution,
(e) a step of dehalogenation of the compound represented by the formula (6) in solution to synthesize a L-thymidine derivative represented by the following formula (7) (wherein R2 and R3 have the same definitions as given above), and
(f) a step of deblocking and crystallization of the compound represented by the formula (7) to synthesize L-thymidine.

2. A process for producing a 2′ position-halogenated L-thymidine derivative, characterized by subjecting 2,2′-anhydro-1-(beta-L-arabinofuranosyl)thymine represented by the following formula (5) to halogenation and subsequent protection, or protection and subsequent halogenation, or protection and simultaneous halogenation to synthesize a 2′ position-halogenated L-thymidine derivative represented by the following formula (6) in solution (wherein R2 and R3 are each independently a protecting group for hydroxyl group, and Y is a halogen atom) and crystallizing said compound in solution to synthesize an L-thymidine derivative represented by the following formula (7) (wherein R2 and R3 have the same definitions as given above).

3. A process for producing a L-thymidine derivative, characterized by subjecting a compound represented by the following formula (6) in solution (wherein R2 and R3 are each independently a protecting group for hydroxyl group, and Y is a halogen atom) to dehalogenation and crystallization, with the proviso that said compound is not isolated from said solution, to synthesize a L-thymidine derivative represented by the following formula (7) wherein R2 and R3 have the same definitions as given above.
